(19) United States
(12) Patent Application Publication
(10) Pub. No.: US 2026/0048092 A1
(43) Pub. Date: Feb. 19, 2026

(54) SYNERGISTIC BOTANICAL FORMULATION FOR THE TREATMENT OF HERPES VIRUS INFECTIONS AND RELATED METHODS

(71) Applicant: Sonoran University of Health Sciences, Tempe, AZ (US)

(72) Inventors: Jeffrey Langland, Chandler, AZ (US); Heather Dirkmaat, Queen Creek, AZ (US); Keely Puchalski, Chandler, AZ (US)

(73) Assignee: Sonoran University of Health Sciences, Tempe, AZ (US)

(21) Appl. No.: 19/304,240

(22) Filed: Aug. 19, 2025

Related U.S. Application Data
(60) Provisional application No. 63/684,848, filed on Aug. 19, 2024.

(51) Int. Cl. A61K 36/185; A61K 36/28; A61K 36/53; A61K 36/536; A61P 31/22

(52) U.S. Cl. CPC A61K 36/185; A61K 36/28; A61K 36/53; A61K 36/536; A61P 31/22

(57) ABSTRACT

A synergistic botanical formulation and related methods for the treatment of herpes virus infections, particularly herpes simplex virus type 1 (HSV-1), are disclosed. The formulation comprises at least one botanical extract with an extracellular antiviral mechanism and at least one botanical extract with an intracellular antiviral mechanism, selected through in vitro assays to provide complementary modes of action. Extracts are prepared under conditions that selectively isolate hydrophilic or hydrophobic constituents, and are combined in proportions based on individual antiviral activity to maximize synergistic inhibition of viral replication. The formulation is standardized to measured biological activity rather than marker compounds, ensuring consistent therapeutic performance despite variability in botanical sources. When administered topically, the formulation reduces lesion healing time, increases antiviral potency compared to conventional over-the-counter treatments, and decreases the likelihood of viral resistance development. Pharmaceutical compositions, methods of preparation, and treatment protocols are also provided.

### CROSS-REFERENCE TO RELATED APPLICATIONS

This application claims the benefit of U.S. Provisional Application No. 63/684,848 entitled “Synergistic Botanical Formulation for the Treatment of Herpes Virus Infection” to Jeffrey Langland et al., filed on Aug. 19, 2024, the contents of which are hereby incorporated by reference in its entirety.

### BACKGROUND

### 1. Field of the Invention

Generally, the invention relates to compositions for the treatment of herpes virus infections.

### 2. Description of Related Art

Herpes simplex virus type 1 (HSV1), and occasionally herpes simplex virus 2, infection are the causative agents of herpes labialis, more commonly known as ‘cold sores’. This virus causes self-limiting painful blistering around the mouth that typically resolve in 7-10 days if left untreated. Current OTC or pharmaceutical topical treatments for ‘cold sores’ have limited efficacy.

So as to reduce the complexity and length of the Detailed Specification, and to fully establish the state of the art in certain areas of technology, Applicant(s) herein expressly incorporate(s) by reference all of the following materials identified in each numbered paragraph below:

Calvert, H. and Geurin, M. (2020). How effective are topical agents for the treatment of cold sores? Evidence-Based Practice 23 (11): 35-36.|DOI: 10.1097/EBP.0000000000000708

Applicant(s) believe(s) that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), Applicant(s) will amend the specification to expressly recite the essential material that is incorporated by reference as allowed by the applicable rules.

### SUMMARY

This invention describes synergistic botanical formulations which target HSV1 at different points in the virus replication cycle and demonstrate more potent antiviral activity in vitro compared to current products on the market. These unique botanical blends provide more rapid resolution of cold sore symptoms and prevent the development of viral resistance to the therapy.

The methodology described in this invention allows for unique botanical formulations to be created based on in vitro assays to provide synergistic therapeutic value. Historically botanical formulations have been created based on their therapeutic value without any laboratory characterization. By doing this, botanicals are often added to a formulation that provide the same mechanism of action or the same active compound. When this is done, the different botanicals in a formulation often do NOT provide any synergistic value because they are providing the same effect. However, this invention provides a novel approach to creating synergistic botanical formulations. By screening botanical extracts using in vitro assays, 1) the mechanism of action of a botanical extract can be determined to a specific target or generalized targets (targeting the virus extracellularly or intracellularly) and/or 2) the active compound or compound characteristics (such as hydrophobic or hydrophilic) can be determined. Then, based on the results of these in vitro assays, botanicals can be combined to create synergistic formulations where the combined botanical extracts target the virus at different steps in the replication process and/or contain structurally different active compounds. This approach modernizes the process of botanical formulation based on initial in vitro characterization rather than creating formulations based solely on anecdotal evidence.

In addition, the methodologies described in this invention allow for the activity of botanical extracts to be standardized to biological activity. Unfortunately, the medicinal value of botanical extracts will often vary based on seasons, geographical location, or other environmental factors. Historically, botanicals are often standardized by a marker compound. A marker compound is a compound typically present in a botanical extract that is easy to measure but may have no role in the therapeutic value of a botanical extract. With the in vitro methodologies described in this invention, botanical extracts can be standardized to a measurable level of biological activity in an in vitro assay (such as antiviral activity). This novel approach allows any botanical extract (from any source, season, etc.) to be standardized to a true measure of biological activity as opposed to a marker compound which may have no related biological role.

The cold sore treatment market shows promising growth prospects driven by rising awareness and increasing incidence rates. Data Bridge Market Research analyses that the Global Cold Sore Treatment Market size was valued at USD 825.24 million in 2022 and is expected to reach USD 1,376.06 million by 2030, registering a CAGR of 6.60% during the forecast period of 2023 to 2030. Consumers have a desire for products with enhanced efficacy and faster relief of cold sores. This invention describes novel botanical-based formulations which provide unique synergistic activity leading to an inhibition in herpes virus replication. When compared to current OTC products on the market, this botanical formulation provides substantially greater antiviral activity in vitro. In addition, this botanical formula reduces the rate of viral resistance and can be standardized to provide consistent activity even when using botanicals.

Implementations of a synergistic botanical formulation for inhibiting herpes virus replication, may comprise at least one botanical extract having an extracellular antiviral mechanism and at least one botanical extract having an intracellular antiviral mechanism, wherein the botanical extracts are present in amounts effective to produce synergistic inhibition of herpes simplex virus type 1 (HSV-1) replication.

Particular aspects may comprise one or more of the following features. The extracellular antiviral mechanism may be determined by inhibition of HSV-1 attachment to host cells as measured by a viral cell binding assay. The intracellular antiviral mechanism may be determined by inhibition of HSV-1 replication post-attachment as measured by a plaque reduction assay. At least one botanical extract may be prepared by extraction in an ethanol solution of about 0-30% by volume to obtain hydrophilic constituents. At least one botanical extract may be prepared by extraction in an ethanol solution of about 60-100% by volume to obtain hydrophobic constituents. The formulation may comprise a first botanical extract having an extracellular antiviral mechanism and hydrophilic active constituents, a second botanical extract having an extracellular antiviral mechanism and hydrophobic active constituents, a third botanical extract having an intracellular antiviral mechanism and hydrophilic active constituents, and a fourth botanical extract having an intracellular antiviral mechanism and hydrophobic active constituents. The botanical extracts may comprise Melissa officinalis, Hamamelis virginiana, Prunella vulgaris, and Tagetes lucida. Each botanical extract may be present in an amount proportional to its individual half-maximal inhibitory concentration (IC50) value against HSV-1 as determined by in vitro antiviral activity assays. The formulation may be standardized based on a measured antiviral biological activity. The percentage of at least one botanical extract may be adjusted to compensate for variability in antiviral activity between different botanical sources. The formulation may exhibit a selectivity index greater than 10 for each botanical extract. The formulation may exhibit at least a 10-fold greater in vitro antiviral activity against HSV-1 than a commercially available over-the-counter cold sore treatment. The formulation may further comprise a pharmaceutically acceptable carrier suitable for topical administration. The pharmaceutically acceptable carrier may comprise a gel base. The gel base may comprise about 50% PCCA Versabase gel.

Implementations of a method of formulating a treatment for a herpes virus infection, may comprise preparing a synergistic botanical formulation by identifying a plurality of botanical extracts having anti-herpes simplex virus type 1 (HSV-1) activity through in vitro assays, selecting at least one botanical extract having an extracellular antiviral mechanism and at least one botanical extract having an intracellular antiviral mechanism, and combining the selected botanical extracts in amounts effective to produce synergistic inhibition of HSV-1 replication.

Particular aspects may comprise one or more of the following features. The formulation may comprise a first botanical extract having an extracellular antiviral mechanism and hydrophilic active constituents, a second botanical extract having an extracellular antiviral mechanism and hydrophobic active constituents, a third botanical extract having an intracellular antiviral mechanism and hydrophilic active constituents, and a fourth botanical extract having an intracellular antiviral mechanism and hydrophobic active constituents. The botanical extracts may comprise Melissa officinalis, Hamamelis virginiana, Prunella vulgaris, and Tagetes lucida.
19. The method of claim 16, wherein the formulation is standardized based on a measured antiviral biological activity. The method may further comprise adjusting the percentage of a botanical extract in the formulation to compensate for variability in antiviral activity between different botanical sources.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventor is fully aware that he can be his own lexicographer if desired. The inventor expressly elects, as his own lexicographer, to use only the plain and ordinary meaning of terms in the specification and claims unless he clearly states otherwise and then further, expressly sets forth the “special” definition of that term and explains how it differs from the plain and ordinary meaning. Absent such clear statements of intent to apply a “special” definition, it is the inventor's intent and desire that the simple, plain and ordinary meaning to the terms be applied to the interpretation of the specification and claims.

The inventor is also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventor is fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventor not to invoke the provisions of 35 U.S.C. § 112(f). Moreover, even if the provisions of 35 U.S.C. § 112(f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

The foregoing and other aspects, features, and advantages will be apparent to those artisans of ordinary skill in the art from the DETAILED DESCRIPTION and DRAWINGS.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 is a chart showing four groupings of HSV1 viral targets of botanical extracts.

FIG. 2 is a diagram modeling botanical extract synergism inhibiting HSV1 replication.

FIG. 3 depicts an example of a synergistic botanical extract formulation.

FIG. 4 is a graph depicting demonstrated synergism of botanical extracts in preventing HSV1 replication.

FIG. 5 is a graph depicting a comparison of a botanical formulation to commercially available anti-viral products.

FIGS. 6A-B provide examples of clinical efficacy of the botanical formulation of FIG. 3 for the treatment of HSV1 associated labialis.

FIG. 7 provides a graphical comparison of anti-HSV-1 activity of various commercial sources of Hamamelis virginiana.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below and it should be noted that while specific products and/or manufacturers may be referred to for exemplary purposes throughout this disclosure, other suitable substitutes are also contemplated.

Herpes simplex virus type 1 (HSV1), and occasionally herpes simplex virus 2, infection are the causative agents of herpes labialis, more commonly known as ‘cold sores’ or ‘fever blisters’. This virus causes self-limiting painful blistering around the mouth that typically resolve in 7-10 days if left untreated. More than 90 percent of all people are infected with the virus by 40 years of age. Primary infection usually occurs in childhood, after which the virus remains latent in the trigeminal ganglion. Recurrence may be triggered by stress factors such as exposure to emotional or physical trauma and fatigue. Two FDA approved drugs for ‘cold sores’ include acyclovir and docosonal. Topical application of acyclovir, a nucleoside analog, has been shown to shorten the duration of active herpes labialis lesions by only 0.6 days. Topical docosonal, the compound present in the OTC medication Abreva, has been shown to shorten the time to resolution of active lesions by only 0.75 days. Botanical remedies have a long history for the treatment of diseases, including viral infections. This invention describes synergistic botanical formulations which target HSV1 at different points in the virus replication cycle and demonstrate more potent antiviral activity in vitro compared to current products on the market.

This invention describes synergistic botanical formulations which target HSV1 at different points in the virus replication cycle and demonstrate more potent antiviral activity in vitro compared to current products on the market.

### Methods

### Botanical Extract Preparation:

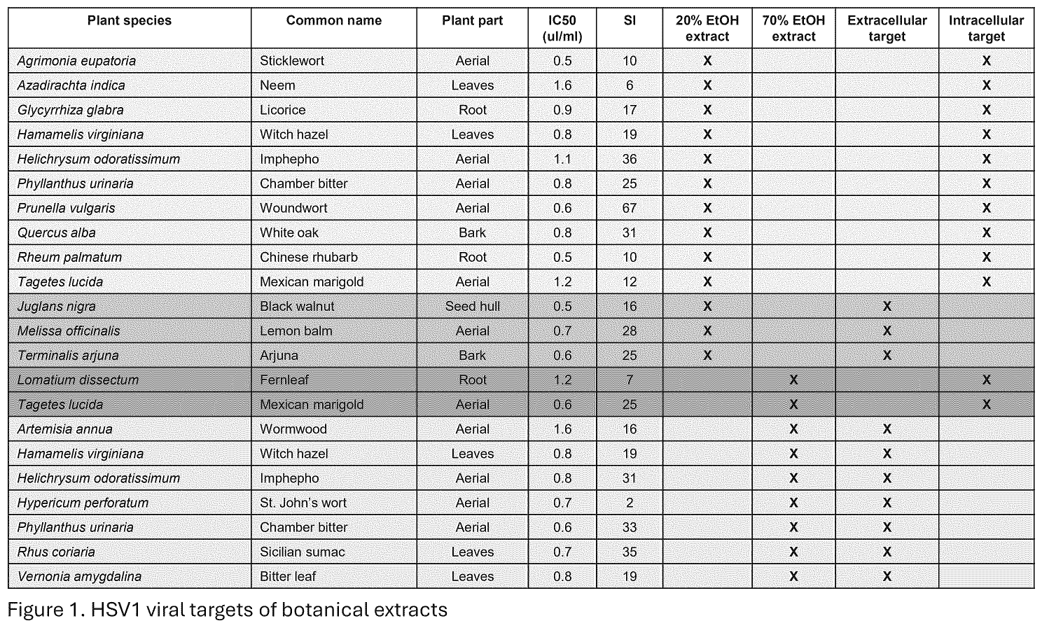

For solvent extraction, dried botanicals (aerial, leaves, roots, bark or seed hull; see FIG. 1) were ground to a fine powder, resuspended in extraction solution (20% or 70% ethanol (EtOH)) and mixed continuously for 24 hours at room temperature. The extract was centrifuged at 3000×G for 10 min to remove large plant debris and the extraction solution filtered through a 0.2 uM filter. For the 20% EtOH extraction, the extract contains more hydrophilic constituents and the extraction could be prepared in 0-30% EtOH. For the 70% EtOH extraction, the extract contains more hydrophobic constituents and the extraction could be prepared in 60-100% EtOH.

### Effect on Virus Replication:

Vero cells (ATCC) were maintained with Dulbecco's Minimal Essential Media (DMEM, Cellgro) supplemented with 100 IU penicillin/ml, 100 ug streptomycin/ml, 2.5 ug amphotericin B/ml, and 10% heat-inactivated fetal bovine serum (HI-FBS, Hyclone). Cells were incubated at 37° C., 5% CO2 in a humidified chamber. Plaque reduction assays were performed by treating 100-200 plaque forming units (pfu) of virus (Herpes Simplex Virus HSV1 KOS strain) with increasing concentrations of the botanical extract or vehicle (20% or 70% ethanol) and infecting cell monolayers for 1 hour at 37° C. Following infection, cells were incubated in media containing an equivalent concentration of the botanical extract or vehicle for 3 days at 37° C. Plaques were visualized by staining with 0.1% crystal violet in 20% methanol. The IC50 (concentration of the extract that inhibits virus replication by 50%) was determined by the dose of the botanical extract that reduced plaque formation by 50%>

### Viral Cell Binding Assays:

The ability of the botanical extract to inhibit viral binding to the cell was performed where Vero cells were infected with 100-200 pfu HSV1 KOS in the presence of increasing concentrations of the botanical extract or vehicle for 2 hours at 4° C. to allow virus binding to the cell. After incubation, cells were washed two times with cold PBS to remove unbound virus, complete media added, and the cells incubated for 3 days at 37° C. followed by crystal violet staining to visualize plaque formation. Those botanical extracts which produced a decrease in plaque formation were identified as extracts that target virus attachment to the cell. Those botanical extracts which did not demonstrate a decrease in plaque formation were identified as extracts that target viral replication post-attachment (viral uptake, intracellular processes, viral assembly, viral release from the cell).

### Selectivity Index:

The cellular toxicity of each botanical extract was determined by treating Vero cells with increasing concentrations of the extract, incubating for 24 hrs at 37° C. followed by standard cell viability testing using an MTS assay kit (Abcam). The CC50 was calculated as the dose of the botanical extract which inhibited cell viability by 50%. The selectivity index was calculated by dividing the CC50 dose by the IC50 dose.

### Clinical Assessment:

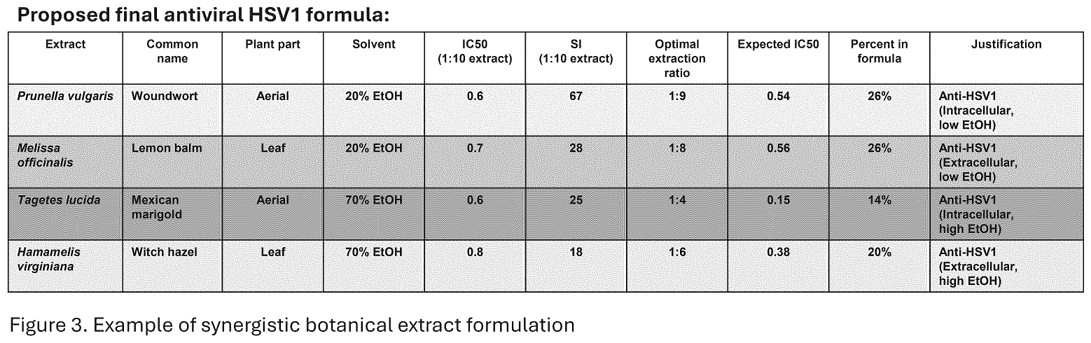

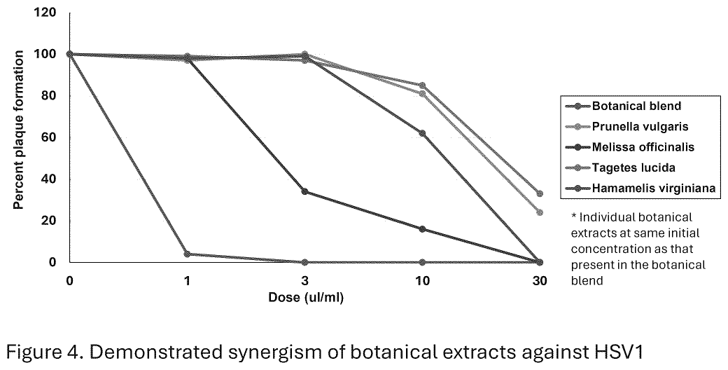

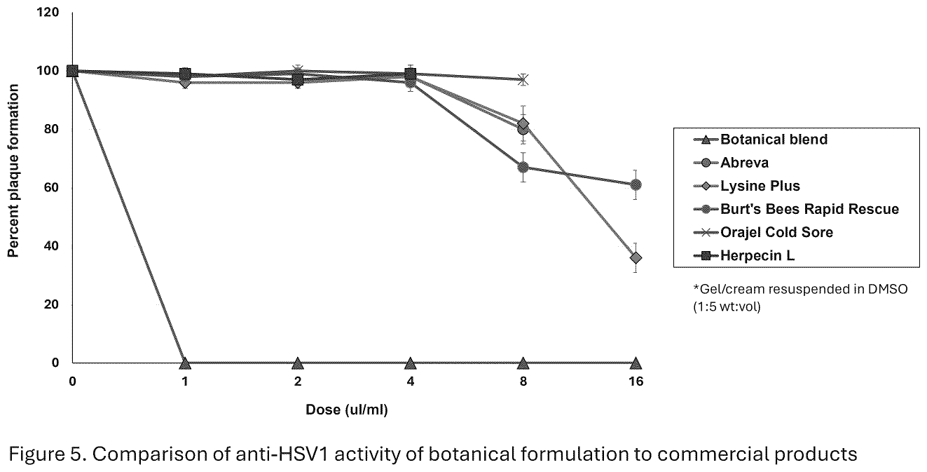

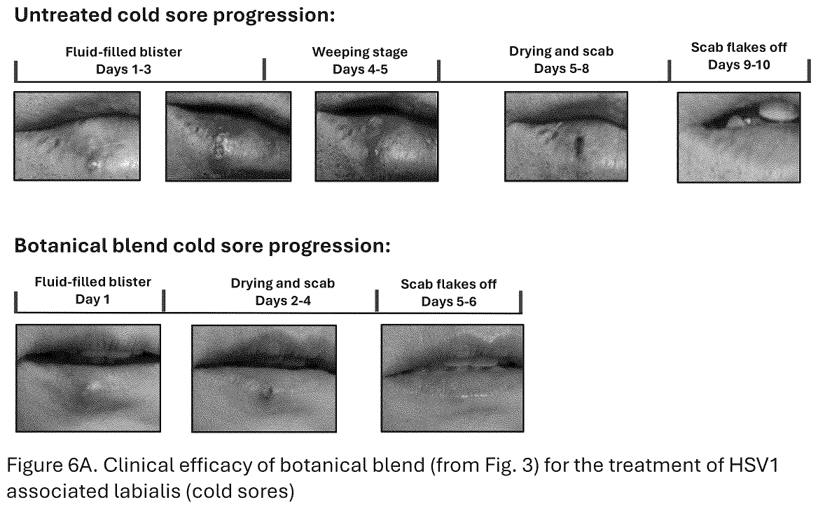

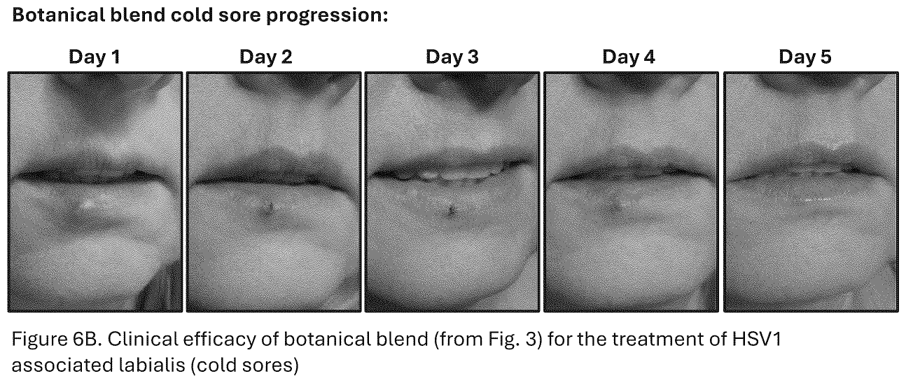

A botanical blend containing Prunella vulgaris, Melissa officinalis, Tagetes lucida and Hamamelis virginiana was prepared (as shown in FIG. 3). The botanical blend solution was resuspended in 50% PCCA Versbase gel for topical application. For treatment, the botanical gel formula was applied topically 5-times daily using a small pea-size amount applied directly onto the lesion. The lesion was photographed daily.

### Results

Twenty-two botanical extracts were found to have antiviral activity against HSV1 as shown in FIG. 1. The activity of these extracts was dependent on the extraction solvent (20% or 70% EtOH) and the part of the plant tested. The active constituents found in plants are secondary metabolites including compounds such as alkaloids, flavonoids, phenolics, glycosides, and saponins. Depending the hydrophobic or hydrophilic nature of these compounds their solubility will vary based on the extraction solvent used. In the case of this work, the more hydrophilic compounds tended to be extracted into the 20% EtOH solvent whereas the more hydrophobic compounds tended to be extracted into the 70% EtOH solvent. In addition, the different botanical extracts were tested for their ability to inhibit HSV1 replication either during the cellular attachment process (Extracellular target) or at a point after viral attachment to the cell (Intracellular target). As shown in FIG. 1, the 22 different botanical extracts were separated based on the EtOH solvent used (20% or 70%) and the viral replication target (Extracellular or Intracellular).

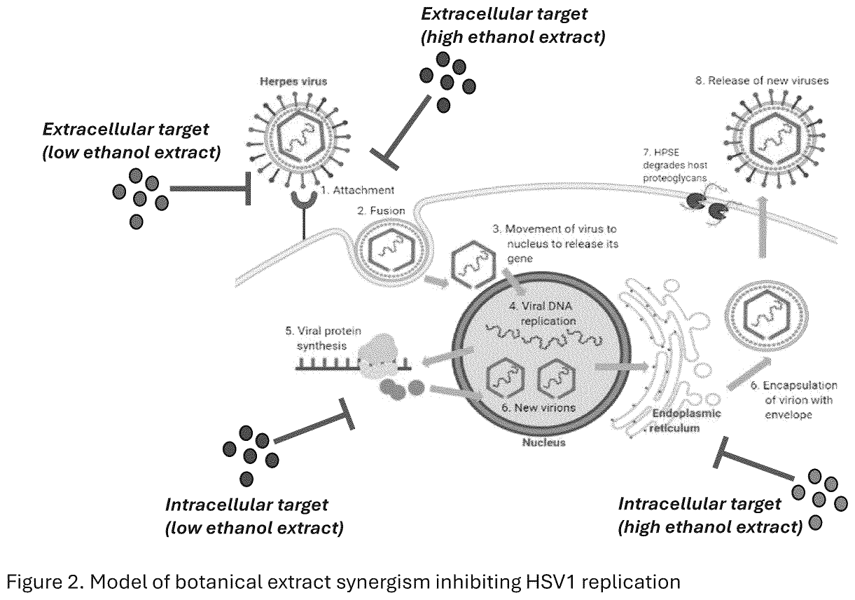

Based on the results from FIG. 1, potential synergistic botanical formulations for targeting HSV1 replication can be formulated. In this case, one botanical extract from each of the four categories is selected and combined together. This combination of four botanical extracts provides two extracts that target the virus extracellularly but with likely different active constituents (one hydrophobic and one hydrophilic) and two extracts that target the virus intracellularly but with likely different active constituents (one hydrophobic and on hydrophilic). The concept behind this synergistic model is shown in FIG. 2 where two extracts, one extracted in low EtOH and one extracted in high EtOH, target the virus extracellularly and two other extracts, one extracted in low EtOH and one extracted in high EtOH, target the virus intracellularly. This concept, therapeutically, will create a botanical formulation that should be able to inhibit HSV1 replication more rapidly leading to an improved rate of resolution of symptoms. In addition, viral resistance is known to commonly occur when using single therapeutic compounds. This synergistic combination of botanical extracts with different active constitutent and with different mechanisms of action reduces or eliminates the virus' ability to develop resistance.

The selectivity index (SI) is a ratio between cytotoxicity and antiviral activity of a drug, or in this case, a botanical extract. The higher the SI ratio, the theoretically more effective and safe a therapeutic is during in vivo treatment for a given viral infection. Typically for botanical extracts, relatively low of SI (<1) means the sample could be toxic and should not be used as a therapeutic. If the calculated SI value is between 1 and 10, some concern is warranted and may be re-evaluated using other biosystems. Calculated SI values>10 are desirable and have more potential therapeutic value. As shown in FIG. 1, for most of the botanical extracts tested, SI values of >10 were observed (19 out of 22 tested). For three of the extracts, SI values between 1-10 were observed. These results suggest that most of the botanical extracts tested have warranted therapeutical value which can be further enhanced by combining select extracts into a synergistic formulations.

FIG. 3 represents a sample of a four-botanical synergistic formulation that could be created based on the activity of the botanicals shown in FIG. 1. This example has two botanicals which target HSV1 extracellularly but prepared in 20% EtOH or 70% EtOH (Melissa officinalis and Hamamelis virginiana, respectively), and two botanicals which target HSV1 intracellularly but prepared in 20% EtOH or 70% EtOH (Prunella vulgaris and Tagetes lucida, respectfully). The four botanicals are combined together based on their individual IC50 activity such that each botanical provides a similar level of antiviral activity in the final formula. The botanical extracts present in this sample formula (FIG. 3) were tested for activity against HSV1 individually and as a complete synergistic blend. As shown in FIG. 4, each of the individual botanical extracts has activity against HSV1, but the combined blend of the four botanical extracts had dramatically increased activity due to the synergistic nature of the combined extracts.

The disclosed synergistic botanical formulations are being developed as a potent therapeutics for the treatment of herpes virus associated ‘cold sores’. This final product is prepared with four synergistic botanicals suspended in a gel base designed for topical application. The example botanical blend described in FIG. 3 was suspended in a gel base (50% PCCA Versabase gel+50% of the four botanical blend) as a prototype topical formulation. This prototype botanical formulation was then used to compare to other OTC ‘cold sore’ treatments currently on the market. In order to test for anti-HSV1 activity, the various OTC products and the prototype botanical formulation were resuspended at a 1:5 ratio in DMSO (Dimethyl sulfoxide) in order to solubilize the products into a liquid form for in vitro testing. As shown in FIG. 5, the prototype botanical formulation maintained potent antiviral activity, while the OTC products had limited antiviral activity. In comparison, the botanical formulation was at least 20-fold higher in antiviral activity compared to the various OTC products (FIG. 5). It should be noted that for the Orajel Cold Sore product and the Abreva product, cellular toxicity was observed at the highest dose tested (FIG. 5).

For clinical assessment, the prototype gel formula (described in FIG. 5) was used to treat an individual patient diagnosed with herpes labialis. The patient applied the botanical gel 5-times topically directly onto the lesion. As shown in FIGS. 6A and 6B, the patient began treatment at the infection stage of a fluid-filled blister. Following 24 hrs of treatment, the swelling of the lesion had reduced almost completely and the lesion dried and scabbed (FIGS. 6A and 6B). The scab continued to reduce over the next few days and flaked off on Day 5 (FIGS. 6A and 6B). In comparison to the progression of untreated herpes labialis, the fluid-filled blister typically lasts for approximately 3-days followed by a weeping stage when the lesion opens and discharges fluid for 2-days (FIG. 6A). For the botanical treated patient, the fluid-filled blister lasted only one day and the weeping stage did not occur. These results suggest that the botanical blend improved healing time of the herpes lesion by 4-5 days.

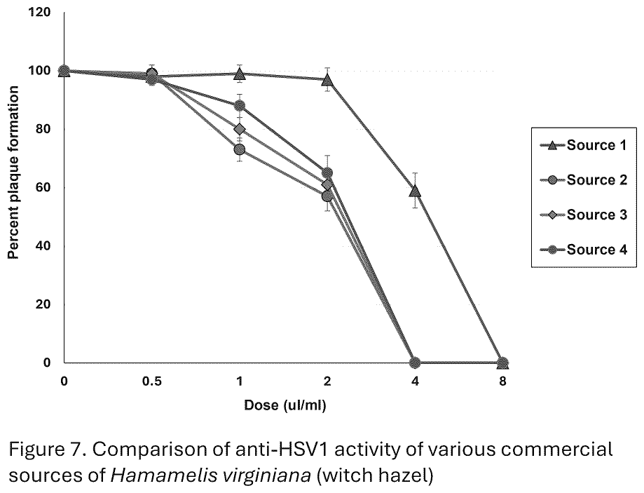

When developing therapeutic products that are formulated using botanical extracts, one concern is consistency of activity from varying sources of the botanical material. The activity of a botanical may vary related to the geographical origin, the time of harvest, seasonal variations, or a number of other factors. The most common industry method for standardization of botanicals is based on the level of specific constituents present in a botanical commonly known as “marker compounds”. Marker compounds often bear little or no relationship to the efficacy of a botanical extract. In this invention, the sourced botanicals are tested for direct antiviral activity against HSV1 and not on the level of marker compounds. Once the antiviral activity of a botanical extract is measured, the percentage of each botanical extract in the final synergistic blend determine by the individual activity of each botanical extract. If a botanical extract contains reduced or no antiviral activity, the percentage of that extract in the final formula may be increased or it would not be used in the final formula. This unique bioassay standardization of botanical extracts provides a method to develop consistent and efficacious botanical remedies. An example of this method of standardization is shown in FIG. 7. Dried Hamamelis virginiana leaves was obtained from four different commercial sources. 70% EtOH extracts were prepared as described in the Methods section and the anti-HSV1 activity was compared between each of the final extracts. As shown in FIG. 7, three of the commercial sources (Source 2, 3, and 4) had similar levels of antiviral activity. However, Source 1 had approximately 50% of the antiviral activity compared to the other sources. For preparation of a final therapeutic formulation for treatment, Source 2, 3, or 4 could be used as normally proposed since they contain the expected/desired level of antiviral activity. For Source 4, if it were to be used in a final therapeutic formula the percentage in the final formula would need to be doubled in order to obtained the desired level of antiviral activity. This methodology allows for botanical formulations for treatment to be standardized to biological activity rather than potentially irrelevant marker compounds.

In places where the description above refers to particular implementations of systems and methods for wound healing and infection reduction, it should be readily apparent that a number of modifications may be made without departing from the spirit thereof and that these implementations may be applied to other to systems and methods for wound healing and infection reduction.

We claim:

1. A synergistic botanical formulation for inhibiting herpes virus replication, comprising:
at least one botanical extract having an extracellular antiviral mechanism; and
at least one botanical extract having an intracellular antiviral mechanism, wherein the botanical extracts are present in amounts effective to produce synergistic inhibition of herpes simplex virus type 1 (HSV-1) replication.

2. The formulation of claim 1, wherein the extracellular antiviral mechanism is determined by inhibition of HSV-1 attachment to host cells as measured by a viral cell binding assay.

3. The formulation of claim 1, wherein the intracellular antiviral mechanism is determined by inhibition of HSV-1 replication post-attachment as measured by a plaque reduction assay.

4. The formulation of claim 1, wherein at least one botanical extract is prepared by extraction in an ethanol solution of about 0-30% by volume to obtain hydrophilic constituents.

5. The formulation of claim 1, wherein at least one botanical extract is prepared by extraction in an ethanol solution of about 60-100% by volume to obtain hydrophobic constituents.

6. The formulation of claim 1, wherein the formulation comprises:
a first botanical extract having an extracellular antiviral mechanism and hydrophilic active constituents;
a second botanical extract having an extracellular antiviral mechanism and hydrophobic active constituents;
a third botanical extract having an intracellular antiviral mechanism and hydrophilic active constituents; and
a fourth botanical extract having an intracellular antiviral mechanism and hydrophobic active constituents.

7. The formulation of claim 6, wherein the botanical extracts comprise Melissa officinalis, Hamamelis virginiana, Prunella vulgaris, and Tagetes lucida.

8. The formulation of claim 1, wherein each botanical extract is present in an amount proportional to its individual half-maximal inhibitory concentration (IC50) value against HSV-1 as determined by in vitro antiviral activity assays.

9. The formulation of claim 1, wherein the formulation is standardized based on a measured antiviral biological activity.

10. The formulation of claim 9, wherein the percentage of at least one botanical extract is adjusted to compensate for variability in antiviral activity between different botanical sources.

11. The formulation of claim 1, wherein the formulation exhibits a selectivity index greater than 10 for each botanical extract.

12. The formulation of claim 1, wherein the formulation exhibits at least a 10-fold greater in vitro antiviral activity against HSV-1 than a commercially available over-the-counter cold sore treatment.

13. The formulation of claim 1, further comprising a pharmaceutically acceptable carrier suitable for topical administration.

14. The formulation of claim 13, wherein the pharmaceutically acceptable carrier comprises a gel base.

15. The formulation of claim 14, wherein the gel base comprises about 50% PCCA Versabase gel.

16. A method of formulating a treatment for a herpes virus infection, comprising:
preparing a synergistic botanical formulation by:
identifying a plurality of botanical extracts having anti-herpes simplex virus type 1 (HSV-1) activity through in vitro assays;
selecting at least one botanical extract having an extracellular antiviral mechanism and at least one botanical extract having an intracellular antiviral mechanism; and
combining the selected botanical extracts in amounts effective to produce synergistic inhibition of HSV-1 replication.

17. The method of claim 16, wherein the formulation comprises:
a first botanical extract having an extracellular antiviral mechanism and hydrophilic active constituents;
a second botanical extract having an extracellular antiviral mechanism and hydrophobic active constituents;
a third botanical extract having an intracellular antiviral mechanism and hydrophilic active constituents; and
a fourth botanical extract having an intracellular antiviral mechanism and hydrophobic active constituents.

18. The method of claim 17, wherein the botanical extracts comprise Melissa officinalis, Hamamelis virginiana, Prunella vulgaris, and Tagetes lucida.

19. The method of claim 16, wherein the formulation is standardized based on a measured antiviral biological activity.

20. The method of claim 19, further comprising adjusting the percentage of a botanical extract in the formulation to compensate for variability in antiviral activity between different botanical sources.
